(19) United States
(12) Patent Application Publication
(10) Pub. No.: US 2011/0229428 A1
(43) Pub. Date: Sep. 22, 2011

(54) FABRIC SOFTENING COMPOSITIONS AND METHODS

(75) Inventors: Donghui Wu, Bridgewater, NJ (US); Leopold Laitem, Lebanon, NJ (US); Guy Broze, Grace-Hoollogne (BE)

(73) Assignee: Colgate-Palmolive Company

(21) Appl. No.: 13/131,175

(22) PCT Filed: Nov. 25, 2009
(86) PCT No.: PCT/US09/65832

§ 371 Date: May 25, 2011

Related U.S. Application Data
(60) Provisional application No. 61/118,070, filed on Nov. 26, 2008.

(51) Int. Cl. A61K 8/40; C07C 67/00; A61Q 5/12; C11D 3/60; A61Q 5/02

(52) U.S. Cl. 424/70.1; 560/253; 510/515; 510/124

(57) ABSTRACT

The invention provides fabric conditioning compositions, which include as the conditioning agent, one or more quaternary ester ammonium salt compounds, which include a mixture of quaternary mono-, di- and tri-ester ammonium salt components, wherein the amount of quaternary diester ammonium salts is greater than about 40% by weight, and the amount of quaternary triester ammonium salts is less than about 20% by weight based on the total amount of quaternary ester ammonium salts. The composition contains a high level of quaternary diester ammonium salt content, and low level of quaternary triester ammonium salt content.

### CROSS REFERENCE TO RELATED APPLICATIONS

This application claims priority to U.S. Provisional Patent Application No. 61/118,070, which was tiled 26 Nov. 2008, which is incorporated herein by reference in it entirety.

### BACKGROUND OF THE INVENTION

Fabric softening compositions suitable for providing fabric softening and static control benefits are known. Such compositions may contain, as the softening component, quaternized fatty acid ester ammonium salts derived from triethanolamine. The quaternized ester ammonium salts are known as esterquats.

The esterquats are typically produced in a process in which triethanolamine is esterified with fatty acids and the reaction product is then quaternized. The esterquats contain mono-, di- and tri-esters of fatty acids. It is desirable to have a high content of di-estersquats and low level of tri-esterquats in a fabric softening composition.

### SUMMARY OF THE INVENTION

There is provided a quaternary ammonium compound having the following structure:

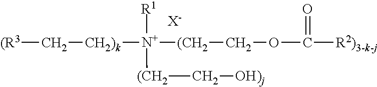

wherein

R¹ is a C₁-C₄ alkyl group, linear or branched:

R² is a C₁₂-C₂₂ alkyl group, linear or branched;

R³ is a short chain ester or alkoxyl group, or a halogen;

X⁻ is a counterion;

j is 0 to (2-k), preferably 0; and

k is between 0 and 2, preferably 1.

The quaternary ammonium compound of the present invention desirably is a mixture of quaternized mono-, di- and tri-esters ammonium compounds, wherein the amount. of quaternized triester ammonium is less than 20% by weight of the total quaternized ester ammonium content, more preferably less than 10% and most preferably less than 5%; and the amount of quaternized diester ammonium is at least 40 weight percent, preferably 60 weight percent, more preferably 80 weight percent of the total quaternized ester ammonium content.

There is provided a method of producing a quaternary ester ammonium salt comprising:

(i) quaternizing and etherizing triethanolamine with an alkylating agent to produce a compound of formula:

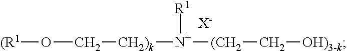

(ii) esterifying above compound to form a compound of formula:

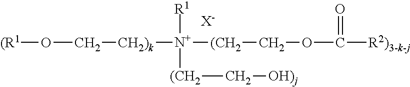

wherein

R¹ is a C₁-C₄ alkyl group, linear or branched;

R² is a C₁₂-C₂₂ alkyl group, linear or branched;

X⁻ is a counterion;

j is 0 to (2-k), preferably 0; and

k is 0 to 2, preferably 1.

There is provided a method of producing an esterquat comprising:

(i) providing a compound of formula:

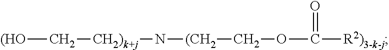

by esterifying triethanolamine with a long chain carboxylic acid,

(ii) providing a compound of the following formula from the above compound:

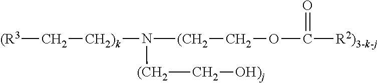

(iii) quote izing the compound of (ii to form the followin compound:

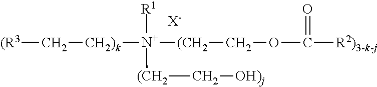

wherein

R¹ is a C₁-C₄ alkyl group, linear or branched;

R² is a C₁₂-C₂₂ alkyl group. linear or branched;

R³ is a short chain alkyl ester, a short chain alkoxyl group, or a halogen;

X⁻ is a counterion;

j is 0 to (2-k), preferably 0; and

k is 0 to 2, preferably 1. The term “short chain” as used herein refers to a chain having from 1 to 4 carbon atoms, linear or branched, unless otherwise indicated.

There is also provide a method of making a quaternary ester ammonium salt comprising:

(i) providing a compound of the formula:

(R³—CH₂—CH₂)ₖ—N—(CH₂—CH₂—OH)₃₋ₖ;

(ii) reacting the above compound to form a compound of the formula:

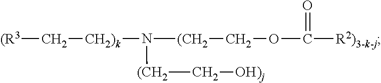

and

(iii) quatemizing the above compound to form a compound of the formula:

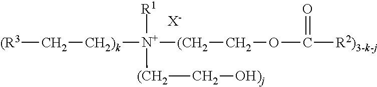

wherein

R¹ is a C₁-C₄ alkyl ciroup, linear or branched;

R² is a C₁₂-C₂₂ alkyl group, linear or branched;

R³ is a short chain ester or a halogen;

X⁻ is a countetion;

j is 0 to 12-k), preferably 0; and

k is 0 to 2, preferably 1.

The invention further provides a fabric softening composition comprising one or more quaternized ester ammonium having mono-, di- and tri-esters, wherein the amount of triester quaternized ester ammonium is less than 20% by weight of the total quaternized ester ammonium content. more preferably less than 10% and most preferably less than 5%.

### DETAILED DESCRIPTION OF THE INVENTION

As used throughout, ranges are used as shorthand for describing each and every value that is within the range. Any value within the range can bc selected as the terminus of the range. In addition, all references cited herein are hereby incorporated by reference in their entireties. In the event of a conflict in a definition in the present disclosure and that of a cited reference, the present disclosure controls.

The present invention is directed to quaternized ester ammonium salt products having high diester ammonium content and low triester ammonium content, as well as adjustable monoester ammonium content. In certain embodiments, the invention is also directed to a textile softening composition, which has as a major ingredient the quaternary diester ammonium salt compound of the invention. The invention also encompasses a textile softening composition, which is non-yellowing and has improved softening performance and desirable textile softening properties such as improved softener biodegradability, viscosity, water absorbency, stability and the like, as well as improved fragrance delivery to textile surface.

The invention encompasses compounds comprising and methods for producing quaternary ester ammonium salts. In particular, desired embodiments encompass an additional reaction step before or after traditional triethanolamine's esterification process that utilizes a long chain fatty acid, preferably a fatty acid having 12-22 carbon atoms. The invention also encompasses a synthesis to produce modified quaternary ester ammonium salts by quaternizing triethanolamine and protecting one or two of its hydroxylgroups first with, for example, an ether bond, in one single step, then proceeding to an esterification process with long chain fa acids to produce final product. The process preferentially produce a reaction product having a high content of quaternary diester ammonium salts. The end results are that fabric softener formulations using the active ingredients have improved softening and fragrance delivery efficacy, as well as improved stability.

The invention is directed to quaternary ester ammonium salt compounds having the following structure:

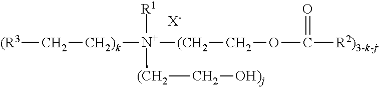

wherein:

R¹ is a C₁-C₄ alkyl group, linear or branched chain;

R² is a C₁₂-C₂₂ alkyl group, linear or branched;

R³ is an ester or alkoxyl group, or a halogen, preferably a short chain ester or alkoxyl group;

X⁻ is a counterion;

j is 0 to (2-k). preferably 0; and

k is 0 to 2, preferably 1.

The quaternary ammonium compound of the present invention desirably is a mixture of quaternized mono-, di- and tri-esters ammonium compounds, wherein the amount of quaternized triester ammonium compounds, which can be characterized as having (3-k-j) equals to 3, is less than 20% by weight of the total quaternized ester ammonium content, more preferably less than 10% and most preferably less than 5%; and the amount of quaternized diester ammonium compounds, which can be characterized as having (3-k-j) equals to 2, is at least 40 weight percent, preferably 60 weight percent, more preferably 80 weight percent of the total quaternized ester ammonium content.

In certain embodiments, the invention is directed to textile softening compositions that comprise one or more of quaternary ester ammonium of this invention. Other embodiments of the invention are directed to a method of making textile softening compositions that have one or more of quaternary ester ammonium salts of this invention.

In certain embodiments. the present invention is directed to a mixture of quaternary ester ammonium salt compounds having the following structure:

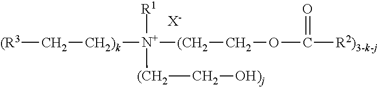

wherein:

R¹ is a C₁-C₄ alkyl group, linear or branched;

R² is a C₁₂-C₂₂ alkyl group;

R³ is a short chain ester or alkoxyl group, or a halogen;

X⁻ is a counterion;

wherein j is 0 to (2-k), preferably 0; and

k is 0 to 2, preferably 1, wherein at least 40 weight percent, preferably 60 weight percent, more preferably 80 weight percent, based on the total weight of the quaternized ester ammonium compounds, has k=1 and j=0.

In various embodiments, R¹ is methyl, ethyl, propyl or butyl, R² is alkyl. both fully saturated or unsaturated chains, R³ may be a halogen such as, e.g., F, and may be Cl, Br, or I; or a short chain ester of formula R—C(O)—O— or alkoxyl of formula R—O—, wherein R is a C₁-C₄ alkyl, such as, e.g., methyl, ethyl, propyl or butyl. In various embodiments, X⁻ is chloride, bromide, fluoride, iodine, CH₃SO₄⁻, or C₂H₅SO₄⁻.

In an exemplary embodiment, R¹ is methyl, X⁻ is chloride, k is 2 and j is 0. In another exemplary embodiment, R¹ is methyl, X⁻ is chloride, k is 1 and j is 0.

In certain embodiments, the present invention is directed to a fabric softener composition comprising one or more quaternary ester ammonium salt compounds described herein.

In other embodiments, the present invention is directed to a method of making a quaternary ester ammonium salt of the invention comprising the steps of:

(i) quatemizing and etherizing triethanolamine with an alkylating agent under conditions suitable to produce a cuatemary ether ammonium salt compound of formula:

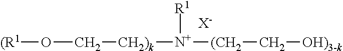

(ii) then esterifying the above compound under conditions suitable to form a compound of formula:

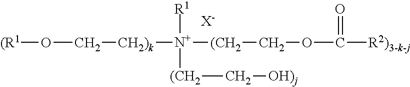

wherein

R¹ is a C₁-C₄ alkyl group;

R² is a C₁₂-C₂₀ alkyl group;

X⁻ is a counterion;

j is 0 to (2-k), preferably 0; and

k is 0 to 2. prefer to be 1.

The quatemizinu and etherizing step is conducted with a short chain alkylating agent having C₁-C₄ carbon atoms. straight or branched, in accordance with conventional csteramine quaternizing reaction processes and conditions. Suitable alkylating agents include alkyl chloride, alkyl bromide, alkyl iodide, and diakyl sulfate, such as, methyl chloride, methyl bromide, methyl iodide, ethyl chloride, ethyl bromide, ethyl iodide, dimethyl sulfate and diethyl sulfate. Of these, more preferred are methyl chloride and dimethyl sulfate. Base, such as sodium bicarbonate, sodium hydroxide, or potassium hydroxide, maybe introduced after quaternizing step to drive the etherizing reaction to completion by removing acid produced from the reaction.

In certain embodiments, the present invention is directed to a method of producing a quaternized ester ammonium salt of the invention comprising:

(i) esterifying, or halogenating triethanolamine under conditions suitable to produce a compound of the following formula by reacting triethanolamine with a short chain carboxylic acid or a halogen compound, for example, thionyl chloride or phosphorous trichloride, or hydrochloric acid in the presence of a catalyst, such as zinc chloride:

(R³—CH₂—CH₂)ₖ—N—(CH₂—CH₂—OH)₃₋ₖ;

(ii) esterifying the above compound with a long chain carboxylic acid, such as a fatty acid, under conditions suitable to form a compound of formula:

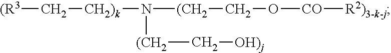

and

(iii) quaternizing with an alkylating agent to form the following compound:

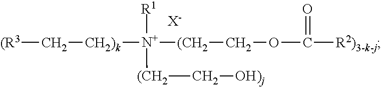

wherein:

R¹ is a C₁-C₄ alkyl group;

R² is a C₁₂-C₂₂ alkyl group;

R³ is a short chain ester or a halotzen;

X⁻ is a counterion;

wherein j is 0 to (2-k), preferably 0; and

k is 0 to 2, preferably 1.

When R′ is an ester, triethanolamine is first reacted with a short chain carboxylic acid having C₁-C₄ carbon atoms, straight or branched, to form an ester in accordance with conventional esteritication reaction process and conditions. When R³ is a halogen, triethanolamine is halogenated with a halogen compound such as the halogen compounds described above, to form a halogenated compound in accordance with conventional halogenation reaction process and conditions.

In a preferred embodiment, thc present invention is directed to a method of producing an quaternary ester ammonium salt of the invention comprising:

(i) reacting triethanolamine with a group capable of forming a compound of formula:

R³—CH₂—CH₂—N—(CH₂—CH₂—OH)₂;

(i) reacting the above compound under conditions suitable to form a compound of formula:

R³—CH₂—CH₂—N—(CH₂—CH₂—O—C(O)—R²)₂; and

(iii) contacting the above compound with an alkylating agent under conditions suitable to form a compound of formula:

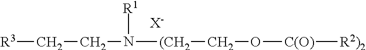

wherein

R¹ is a C₁-C₄ alkyl group;

R² is a C₁₂-C₂₂ alkyl group;

R³ is a short chain ester, or a halogen; and

X⁻ is a counterion.

The compound of formula R³—CH₂—CH₂—N—(CH₂—CH—OH)₂ is preferentially produced by controlling the reactant mole ratio between triethanolamine and the reactant group that forms R³. For example, from 1.2:1 to 1:1 mole ratio of triethanolamine and acetic. acid can be utilized to preferentially produce CH₃—CO—O—CH₂—CH₂—N—(CH₂—CH₂—OH)₂. Utilizing such a compound having only two reactive moieties, i.e., the hydroxyl moieties, ensures that the following esterffleation step with a long chain carboxylic acid, e.g., a fatty acid, ensures that significant amounts of triester amine are not produced. The resulting quaternized product of the esterified amine compound has no or a low level olquaternary triester ammonium.

In various embodiments, R¹ is methyl, ethyl, propyl or butyl; R² is C₁₂-C₂₂ alkyl, linear or branched; and R³ is a halogen including, but not limited to F, Cl, Br, or I. In another embodiment, R³ is short chain ester of formula R—C(O)—O—, wherein R is a C₁-C₄ alkyl. In another embodiment, X⁻ is a halogen including, but not limited to, F⁻, Cl⁻, Br⁻, or I⁻. In an exemplary embodiment, R¹ is methyl, R³ is a —O—C(O)CH₃, and X⁻ is chloride. In another exemplary embodiment, R¹ is methyl, R³ is chlorine. and X⁻ is chloride.

In certain embodiments, the present invention is directed to a method of producing an esterquat or quaternary ester ammonium salt compound of the invention comprising:

(i) esterifying triethanolamine with a group capable of forming an ester, such as a carboxylic acid having alkyl group, e.g., a fatty acid, under conditions suitable to produce a compound of formula:

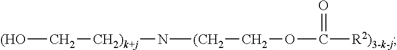

(ii) modifying the above compound under conditions suitable to form a compound of formula:

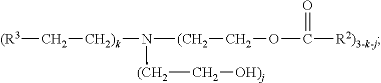

(iii) quaternizing with an alkylating agent to form the following compound:

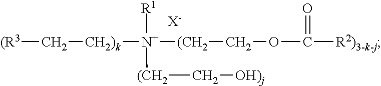

wherein:

R¹ is a C₁-C₄ alkyl group;

R² is a C₁₂-C₂₂ alkyl group;

R³ is a short chain ester or alkoxyl group, or a halogen;

X⁻ is a counterion;

wherein j is 0 to (2-k), preferably 0; and

k is 0 to 2, preferably 1. The initial esteritYing step preferentially provides diesteramines and minimizes the content of triesteramines by controlling the molar ratio between the carboxylic acid and triethanolamine. Desirably. the ratio is less than 2.2, preferably between 2 and 1.5. and more preferably between 1.6 and 1.8.

In various embodiments, R¹ is methyl, ethyl, propyl or butyl, either straight or branched chain; and R² is C₁₂₋₂₂ alkyl, either saturated or unsaturated. In various embodiments, R³ is an alkoxyl or a halogen including, but not limited to, F, Cl, Br, or I.

In various embodiments. R³ is a short chain ester of formula R—C(O)—O—, wherein R is a C₁-C₄ alkyl such as, e.g., methyl, ethyl, propyl or butyl. In various embodiments, k is 0, 1 or 2 and j is 1 or 2. In various embodiments, X⁻ is chloride. In an exemplary embodiment. R^{I} is methyl, X⁻ is chloride, k is 2 and j is 0. In a desired embodiment, R¹ is methyl, X⁻ is chloride. k is 1 and j is 0.

The reference to esterification reaction in the present invention is typically carried out in a temperature between 175° C. and 210° C. with an acid catalyst, such as sulfonic acid, phosphorous acid, p-toluene sulfonic acid or an acceptable Lewis acid. The halogenation reaction is typically carried out by reacting triethanolamine with hydrochloric acid in the presence of a catalyst, such as zinc chloride. Alternatively, triethanolamine can be reacted with thionyl chloride or phosphorous trichloride directly. The quaternization is typically carried out in bulk or in solvent at a temperature between 60° C. and 120° C. Suitable solvents for the quaternization reaction include polar solvents such as lower alcohols (such as isopropyl alcohol) and glycols.

Suitable long chain carboxylic acids for the present invention include fatty acids having 12 to 22 carbon atoms. Preferred fatty acids include, but are not limited to, oleic, palmitic, erucidic, eicosanic, stearic, myristic and mixtures thereof Soy, tallow, palm, palm kernel, rape seed, lard, and mixtures thereof are typical sources for fatty acids which are suitable for the present invention.

Quaternary ester ammonium salt compounds or esterquats of the invention provide softening and antistatic benefits upon application on fabric. The esterquats of the invention may form vesicles in fabric softener compositions, and the size and surface charges of these vesicles may affect the efficacy of the esterduat delivery to fabric surfaces upon application. Vesicles deliver fragrance in softener compositions. because they encapsulate fragrance oils inside the vesicle structures.

In certain embodiments, the present invention is also directed to methods to modify the production of triethanolamine based esterquats. The resulting modified esterquats of the invention have improved softener efficacy and enhanced fragrance delivery.

In certain embodiments, the present invention is also directed textile softener compositions based on esterquats from present invention. In various embodiments, the compositions may contain the esterquat accordingto the present invention, in an amount from 5% to 50%, from 7.5% to 40%, from 10% to 35%, or from 12.5% to 30%; fragrance in an amount from 0.1% to 10%, from 0.25 to 7.5%, or from 0.5 to 5%; water and may also contain small amounts of electrolyte. In certain embodiments, the compositions may contain cationic polymer as rheology modifier, in amounts from 0.01% to 1%, from 0.05% to 0.75%, or from 0.1% to 0.5%; as well as silicone polymer for softening effects. These compositions can be prepared based on traditional methods that are familiar to a person having ordinary skill in the art.

The softener composition of the present invention has a high content of diesterquats and a low content of triesterquats.

The following are non-limiting examples that describe certain embodiments of the present invention.

### EXAMPLES

### Example 1

A triethanolamine based esterquat of the invention is produced from following reactions:

N—(CH₂—CH₂—OH)₃+x R—COOH→R—C(O)—O—CH₂—CH₂—N—(CH₂—CH₂—OH)₂ (I)

(R—C(O)—O—CH₂—CH₂)₂—N—CH₂—CH₂—OH (II)

(R—C(O)—O—CH₂—CH₂)₃—N (III)

where R is a C₁₂-C₂₂ alkyl group. and the molar ratio of long chain fatty acid to triethanolamine x is between 1-2.

An additional reaction step can be implemented to replace hydroxyl group(s) n compound I after the initial esterification reaction completes with a group X, such as, but not limited to. a short chain ester (—O—C(O)—R′, R′ group is a short chain alkyl group, such as—CH₃) or a halogen (—F, —Cl, —Br, or —I). Non-limiting examples of additional reaction after the esterification reaction are as follows:

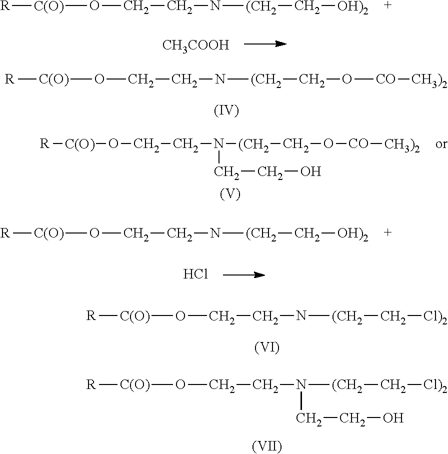

In certain embodiments. Compounds IV-VII go through further reaction with an alkylating agent, for example, methyl chloride, to be quaternized to produce modified esterquats that are different from traditional triethanolamine based esterquats. In the final softener compositions, the end results include surprisingly enhanced softening efficacy and improved fragrance delivery. Various techniques known to one of skill in the art can be substituted, for example, acetic anhydride (CH₃—C(O)—O—C(O)—CH₃) may be used instead of acetic acid and thionyl chloride (SOCl₂) or phosphorous trichloride (PCl₃) may be used instead of hydrochloric acid with a catalyst.

### Example 2

An additional reaction step can be implemented to replace one hydroxyl group in triethanolamine with a functional group X, such as, but not limited to, a short chain ester (—O—CO—R′, R′ group is a short chain alkyl group, such as —CH₃), or a halogen (—F, —Cl, or —Br), before the esterification reaction. The non-limiting examples of the additional reaction before the esterification reaction are:

N—(CH₂—CH₂—OH)₃+CH₃—COOH→CH₃—C(O)—O—CH₂—CH₂—N—(CH₂—CH₂-—OH)₂ (VIII)

(CH₃—C(O)—O—CH₂—CH₂)₂—N—CH₂—CH₂—OH (IX)

(CH₃—C(O)—O—CH₂—CH₂)₃—N (X)

or

N—(CH₂—CH₂—OH)₃+HCl→

Cl—CH₂—CH₂—N—(CH₂—CH₂—OH)₂ (XI)

(Cl—CH₂-—CH2)₂—N—CH₂—CH₂—OH (XII)

(Cl—CH₂—CH₂)₃—N (XIII)

The generic formula of products from reactions 4 and 5 are (where k=1, 2, or 3):

(P—CH₂—CH₂)ₖ—N—(CH₂—CH₁—OH)₃₋ₖ (XIV)

Various techniques can be substituted; for example, acetic anhydride (CH₃—C(O)—O—C(O)—CH₃) may be used instead of acetic acid and thionyl chloride (SOCl₂) or phosphorous trichloride (PCl₃) may be used instead of hydrochloric acid with a catalyst.

By replacing —OH groups on triethanolamine with 1:1 mole of a protective group P, modified triethanolamines can be produced with majorities (more than 50%) as single substitution (e.g., compounds XIV with k=1, or specifically VIII and XI) and small amount of double substitution (e.g., compounds XIV with k=2, or specifically IX and XII). Minimal original unreacted triethanolamine and tri-substitution (e.g., compounds XIV with k=3, or specifically X and XIII) are identified.

The resulting materials (compound XIV with k=1 and 2) from reaction 4 and 5 (except compound XIV with k=3) further go through an esterification process (similar to reaction I) to produce modified diester and monoester, with very minimum amounts of triester (from small amount of unreacted triethanolamine as described in the following reactions (where j=0 to 2-k):

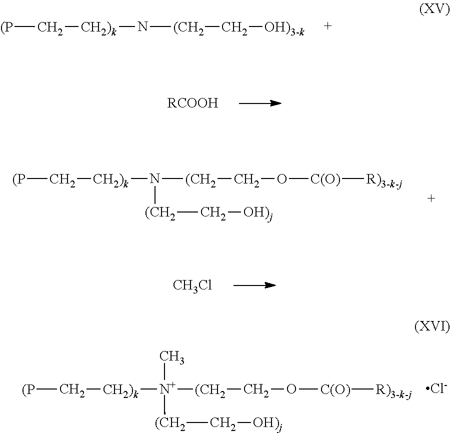

wherein R is a C₁₂-C₂₂ alkyl group, both fully saturated or unsaturated chains can be used.

Compounds XV go through further reaction with all alkylating agent such as methyl chloride to be quaternized to produce modified esterquats, such as those described by compound XVI. As an alternative methyl chloride, dimethyl or diethyl sulfate mar be uses for the quatemization step.

### Example 3

This Example is directed to a new and improved approach to producing modifi esterquats so that benefits, such as improved softening effect and enhanced fragrance delivery, can be obtained. In order to reduce the amount of triesterquat and modify monoesterquat at the same time, the traditional esterquat production process is reversed.

In certain embodiments, instead of esterification first then quaternization, quaternization of amine was done first with quaternization agents, such as, for example, methyl chloride, dimethyl or diethyl sulfate. In certain embodiments, an etherification process occurs when molar ratio of quaternization aent to triethanolamine is higher than 1:1, which converts one or more of the three hydroxyl group(s) on triethanolamine to an ether bond and prevents further reaction with long chain fatty acid to form tri-esterquat. The reactions are described below. To ensure the completion of reaction and minimize undesirable reaction and formation of Compound XX, quaternization agents are slowly added to triethanolamine, and no more than 3:1 mole of alkylating agent was introduced, in which 1 mole of alkylating agent is consumed by quaternization process since it is a preferred reaction. Base, such as sodium bicarbonate, sodium hydroxide, or potassium hydroxide, maybe introduced after quaternizing step to drive the etherizing reaction to completion by removing acid produced from the reaction.

—N—(CH₂—CH₂—OH)₃+CH₃Cl→(N⁺—CH₃)—(CH₂—CH₂—OH)₃Cl⁻ (XVII)

+CH₃Cl→HCl+CH₃—O—CH₂—CH₂—(N⁺—CH₃)—(CH₂—CH₂—OH)₂Cl⁻ (XVIII)

+CH₃Cl→HCl+(CH₃—O—CH₂)₂—(N⁺—CH₃)—(CH₂—CH₂—OH)Cl⁻ (XIX)

+CH₃Cl→HCl+(CH₃—O—CH₂—CH₂)ₖ—(N⁺—CH₃)Cl⁻ (XX)

The generic formula of products from the above reactions are (where k=0, 1, 2, 3, respectively):

(CH₃—O—CH₂—CH₂)ₖ—(N⁺—CH₃)—(CH₂—CH₂—OH)₃₋ₖCl− (XXI)

In certain situations, the quaternization reaction occurs before the etheritication process, methyl-triethanoi-ammonium chloride (compound XVII) will form first. With the introduction of additional methyl chloride, ether bonds will start to form. When the molar ratio of methyl chloride to triethanolamine is controlled to be at or slightly hiaher than 2:1 and methyl chloride is slowly added to triethandamine, the majority of the products are methyl-methoxyiethyl-diethanol ammonium chloride (compound XVIII) with small amount of methyl-triethanol-ammonium chloride (compound XVII) and methyl-dimethoxylethyl-ethanol ammonium chloride (compound XIX), and minimum amount of undesirable methy trimethoxylethyl-ammonium chloride (compound XX).

The resulting materials (compound XXI with k=0, 1 and 2), except compound XX with k=3, further go through an esterification process to produce modified di-ester and mono-ester with very minimum amount of tri-ester (from small amount of unreacted triethanolamine and methyl-triethanol-ammonium chloride compound XVII). The esterification process is described in the following reaction (where j=0 to 2-k):

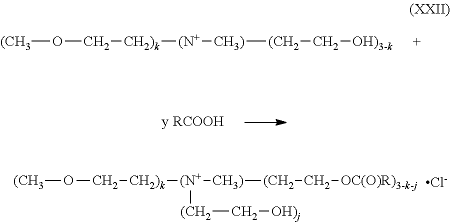

Under certain circumstances, the molar ratio of long chain fatty acid to compound XXI, y may depend on the composition of compound XXI. Compounds XXII are already quaternized in the previous reaction step. Therefore, no further reaction is necessary. The reaction products are mainly modified mono- and di-esterquats when compared to traditional process to product esterquat from triethanolamine. Only a very small amount of traditional tri-esterquats are produced from unreacted triethanol-amine or ammonium chloride. The levels are significantly lower than the traditional process.

### Example 4

This Example is directed to textile softener compositions based on esterquats from present invention. The compositions contain modified esterquat in current invention, in amounts of about 5% to 50%; fragrance in amounts of about 0.1% to about 10%; a small amount of electrolyte, and water. The compositions may contain cationic polymer as rheology modifier, in amounts of about 0.01% to about 1%; and silicone polymer for softening effects. Examples of compositions for this invention are listed in Table 1. These compositions can be prepared based on traditional methods that are familiar to a person having ordinary skill in the art.

TABLE 1
Compositions of Textile Softener

| | | Cationic | | |
|---|---|---|---|---|
| | | polymer- | | |
| | Modified | rheology | Silicone | |
| Composition | esterquat | modifiers | polymer | Fragrance |
| #1 | 15% | None | None | 1-5% |
| #2 | 15% | 0.1% | None | 1-5% |
| #3 | 15% | None | 1% | 1-5% |

The present invention is not to be limited in scope by the specific embodiments disclosed in the Examples, which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

The compositions and methods of the present invention are applicable for many potential consumer products, including, without limitation, fabric softeners, fabric conditioners, laundry detergents and other household cleaners, as well as personal care applications such as hair care products, e.g., shampoos, moisturizers and conditioners.

What is claimed is:

1. A composition comprising a mixture of quaternary ester ammonium compounds having the following structure: 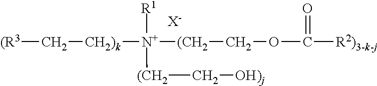
wherein
R¹ is a C₁-C₄ alkyl group, linear or branched;
R² is a C₁₂-C₂₂ alkyl group, linear or branched;
R³ is a halogen, or an ester or alkoxyl group comprising 1 to 4 carbon atoms;
X⁻ is a counterion;
j is 0 to (2-k); and
k is 0 to 2;
wherein the mixture comprises quaternized mono-, di- and tri-ester ammonium compounds having the structure, and wherein the amount of the quaternized triester ammonium compound is less than 10% by weight of the mixture comprising the quaternized mono-, di- and tri-ester ammonium compounds.

2. The composition of claim 1, wherein k is 1 or 2.

3. The composition of claim 1, wherein j is 0, 1, or 2.

4. The composition of claim 1, wherein X⁻ is chloride or methyl sulfate.

5. The composition of claim 1, wherein R¹ is methyl, X⁻ is chloride or methyl sulfate, k is 2 and j is 0.

6. The composition of claim 1, wherein R¹ is methyl, X⁻ is chloride or methyl sulfate, k is 1 and j is 0.

7. The composition of claim 1, wherein R¹ is methyl, X⁻ is chloride or methyl sulfate, k is 1 and j is 1.

8. The composition of claim 1, wherein the amount of the quaternized diester ammonium compound is at least 60% by weight of the mixture comprising the quaternized mono-, di- and tri-ester ammonium compounds.

9. The composition of claim 8, wherein the amount of the quaternized diester ammonium compound is at least 80% by weight of the mixture comprising the quaternized mono-, di- and tri-ester ammonium compounds.

10. The composition of claim 8, wherein the amount of the quaternized triester ammonium compound is less than 5% by weight of the mixture comprising the quaternized mono-, di- and tri-ester ammonium compounds.

11. A fabric softener composition comprising the composition of claim 1.

12. A method of producing a quaternary ester ammonium compound comprising:
(i) reacting triethanolamine with an alkylating agent under conditions suitable to produce a compound of formula: 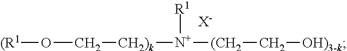
(ii) reacting the above compound with a carboxylic acid under conditions suitable to form a compound of formula: 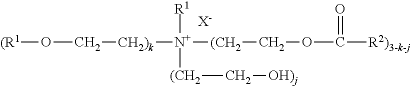
wherein
R¹ is a C₁-C₄ alkyl group, linear or branched;
R² is a C₁₂-C₂₂ alkyl group, linear or branched;
X⁻ is a counterion;
j is 0 to (2-k); and
k is 0 to 2.

13. The method of claim 12, wherein k is 1 or 2.

14. The method of claim 12, wherein j is 0, 1 or 2.

15. The method of claim 12, wherein X⁻ is chloride or methyl sulfate.

16. The method of claim 12, wherein R¹ is methyl, X⁻ is chloride or methyl sulfate, k is 2 and j is 0.

17. The method of claim 12, wherein R¹ is methyl, X⁻ is chloride or methyl sulfate, k is 1 and j is 0.

18. The method of claim 13, wherein R¹ is methyl, X⁻ is chloride or methyl sulfate, k is 1 and j is 1.

19. A method of producing a quaternary ester ammonium compound comprising:
(i) reacting triethanolamine with a carboxylic acid under conditions suitable to produce compounds of formula: 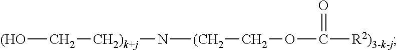
(ii) reacting the above compound under conditions suitable to form compounds of formula: 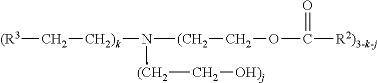
(iii) reacting with an alkylating agent to form the following compounds: 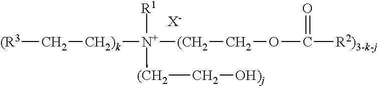
wherein
R¹ is a C₁-C₄ alkyl group, linear or branched;
R² is a C₁₂-C₂₂ alkyl group, linear or branched;
R³ is a short chain ester or a halogen;
X⁻ is a counterion;
j is 0 to (2-k); and
k is 0 to 2.

20. A method of producing a quaternary ester ammonium compound comprising:
(i) reacting triethanolamine under conditions suitable to produce compounds of formula:
(R³—CH₂—CH₂)ₖ—N—(CH₂—CH₂—OH)₃₋ₖ;
(ii) reacting the above compound under conditions suitable to form compounds of formula: 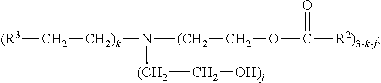
and
(iii) reacting the above compound with an alkylating agent under conditions suitable to form compounds of formula: 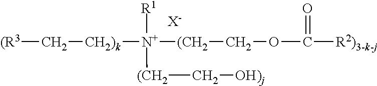
wherein
R¹ is a C₁-C₄ alkyl group, linear or branched;
R² is a C₁₂-C₂₂ alkyl group, linear or branched;
R³ is a short chain ester or a halogen;
X⁻ is a counterion;
j is 0 to (2-k); and
k is 0 to 2.

21. A hair care composition comprising the composition comprising a mixture of quaternary ester ammonium compounds of claim 1.

22. A fabric softening composition comprising the quaternary ester ammonium compound produced by the method of claim 12.

23. A fabric softening composition comprising one or more quaternary ester ammonium compounds, which include a mixture of quaternary mono-, di- and tri-ester ammonium components, wherein the quaternary diester ammonium compound is more than 60% by weight, and the quaternary triester ammonium is less than 10% by weight based on the total weight of quaternary ester ammonium compounds.

24. A fabric softening composition comprising the composition of claim 1, wherein the mixture of quaternary ester ammonium compounds comprises less than 10% by weight of the quaternary ester ammonium compounds having (3-k-j)=3 and more than 60% by weight of the quaternary ester ammonium compounds having (3-k-j)=2, based on the total weight of quaternary ester ammonium compounds.
